(19) United States
(12) Patent Application Publication
(10) Pub. No.: US 2022/0226073 A1
(43) Pub. Date: Jul. 21, 2022

(54) CONTROLLING MOTION OF MAGNETICALLY-DRIVEN MICROSCOPIC PARTICLES

(71) Applicant: INDIAN INSTITUTE OF SCIENCE, Bangalore, Karmataka (IN)

(72) Inventors: Shanmukh Srinivas PEDDI, Bangalore (IN); Debayan DASGUPTA, Bangalore (IN); Ambarish GHOSH, Bangalore (IN)

(21) Appl. No.: 17/613,671

(22) PCT Filed: May 27, 2020
(86) PCT No.: PCT/IN2020/050475

§ 371 Date: Nov. 23, 2021

(30) Foreign Application Priority Data
May 30, 2019 (IN) 201941021608

(51) Int. Cl. A61C 5/50; H01F 5/00; A61C 19/06; A61C 3/00

(52) U.S. Cl. CPC A61C 5/50; A61C 3/00; A61C 19/06; H01F 5/00

(57) ABSTRACT

Devices, systems and methods for controlling motion of magnetic-driven nanobots are provided. Based on a selection indicative of a pattern of movement of the nanobots (200), a signal can be generated indicative of a pattern of magnetic field to be produced. Electrical signals can be generated to cause production of the pattern of magnetic field. The electrical signals can be provided to a device (300, 800) which is adaptable for being placed on the head or around a tooth of the patient. A first coil (502, 602, 804) of the device can receive the electrical signals and produce the pattern of the magnetic field to drive the magnetically-driven nanobots from a pulp region of the tooth into the dentinal tubules.

### TECHNICAL FIELD

The present subject matter relates, in general, to controlling motion of microscopic particles and, in particular, to controlling motion of magnetically-driven nanobots.

### BACKGROUND

Motion of active particles, for example, microscopic particles, such as nanobots, may have to be controlled in some cases. For instance, nanobots may have to be injected into microscopic channels and their motion inside such channels may have to be controlled. Example microscopic channels are dentinal tubules, which extend radially outwards starting from the central root canal space (the chamber which contains the pulp) all the way into the thickness of the dentine tissue tapering in their course from 2 μm to 0.9 μm. Dentinal tubules are in the form of small and hollow canals filled with dentinal fluid which facilitates sensations from the outside of the tooth to the pulpal nervous system. Bacterial species, such as Enterococcus, have been documented to proliferate deep into the dentinal tubules in an infected root canal. To cure bacterial infections, antibacterial agents may be injected into the dentinal tubules.

### BRIEF DESCRIPTION OF DRAWINGS

The features, aspects, and advantages of the present subject matter will be better understood with regard to the following description and accompanying figures. The use of the same reference number in different figures indicates similar or identical features and components.

FIG. 1 illustrates a horizontal section of a sample of a tooth, in accordance with an example implementation of the present subject matter.

FIG. 2 illustrates a nanobot, in accordance with an example implementation of the present subject matter.

FIG. 3 depicts a cap structure for causing movement of magnetically-driven nanobots into dentinal tubules of a tooth of a patient, in accordance with an example implementation of the present subject matter.

FIG. 4 depicts an example coil arrangement, in accordance with an example implementation of the present subject matter.

FIG. 5 depicts another example coil arrangement, in accordance with an example implementation of the present subject matter.

FIG. 6 depicts another example coil arrangement, in accordance with an example implementation of the present subject matter.

FIG. 7 depicts the cap structure placed around the tooth, in accordance with an example implementation of the present subject matter.

FIG. 8(a) depicts an example helmet adapted for placement on the head of the patient, in accordance with an example implementation of the present subject matter.

FIG. 8(b) depicts a coil arrangement, in accordance with an example implementation of the present subject matter.

FIG. 9(a) depicts block diagram of a system to cause movement of magnetically-driven nanobots into dentinal tubules of a tooth of a patient, in accordance with an example implementation of the present subject matter.

FIG. 9(b) depicts an example arrangement of the system of FIG. 9(a), in accordance with an example implementation of the present subject matter.

FIGS. 10(a) and 10(b) illustrate movement of a nanobot under the influence of an oscillating magnetic field, in accordance with an example implementation of the present subject matter.

FIG. 10(c) depicts effective displacement of nanobot under the oscillating magnetic field, in accordance with an example implementation of the present subject matter.

FIG. 10(d) illustrates trajectories followed by three nanobots under the influence of an oscillating magnetic field, in accordance with an example implementation of the present subject matter.

FIG. 11 illustrates the motion of nanobots under the influence of a combination of an oscillating magnetic field and a gradient magnetic field, in accordance with an example implementation of the present subject matter.

FIG. 12(a) illustrates the motion of nanobots under the influence of a combination of a rotating magnetic field and a gradient magnetic field, in accordance with an example implementation of the present subject matter.

FIG. 12(b) illustrates motion of nanobots under the influence of the rotating magnetic field, in accordance with an example implementation of the present subject matter.

FIG. 12(c) illustrates motion of a nanobot under the influence of a revolving rotating magnetic field, in accordance with an example implementation of the present subject matter.

FIG. 13 illustrates motion of a nanobot under the influence of an elliptical magnetic field, in accordance with an example implementation of the present subject matter.

FIG. 14 illustrates subjecting nanobots under a combination of an oscillating magnetic field and a gradient magnetic field, in accordance with an example implementation of the present subject matter.

FIG. 15 illustrates the movement of nanobots under the influence of a combination of an oscillating magnetic field and a DC magnetic field, in accordance with an example implementation of the present subject matter.

FIG. 16(a) illustrates movement of a nanobot under the influence of a slowly changing rotating magnetic field, in accordance with an example implementation of the present subject matter.

FIG. 16(b) illustrates variation of a rotating magnetic field with time, in accordance with an example implementation of the present subject matter.

FIG. 16(c) illustrates a right-handed nanobot, in accordance with an example implementation of the present subject matter.

FIG. 16(d) illustrates a left-handed nanobot in accordance with an example implementation of the present subject matter.

FIG. 17 depicts a method for causing movement of magnetically-driven nanobots comprising an integrated magnetic material into dentinal tubules of a tooth of a patient, in accordance with an example implementation of the present subject matter.

FIG. 18 depicts a Scanning Electron Microscopy (SEM) of a section of the tooth, in accordance with an example implementation of the present subject matter.

### DETAILED DESCRIPTION

Active particles are particles which can be manipulated by remote energy sources. Active particles may be for example, microscopic particles, such as nanobots. Active particles may have to be injected into microscopic channels, such as dentinal tubules. For instance, a dentinal tubule may have to be injected with nanobots having anti-bacterial agents, as will be explained below:

Dentinal tubules are generally about the radial thickness of dentine tissue (up to 2000 μm) in length and have a tapered structure, with the width of the tubule decreasing along its length. For instance, the dentinal tubules may have a diameter of 2.5 μm on one end and 0.9 μm on another end. Bacterial species, such as Enterococcus, have been documented to proliferate in almost the entire length of the dentinal tubules (up to 2000 μm) in an infected root canal.

The complex microscopic nature of the dentinal tubules restricts diffusion and approachability of medicaments in the dentinal tubules. Therefore, existing sterilization protocols may not eliminate bacteria residing deep inside dentinal tubules. Consequently, even after treatment of the tooth, reinfection of the tooth may happen.

Several different techniques have been tried to increase the effective depth to which the antibacterial agents can penetrate. Such techniques include electrochemical methods, ultrasonic activation of antibacterial agents, driving the nanometric antibacterial agents into canal using photoacoustic streaming and ultrasonic devices, and LASER photodynamic therapy. Although the above techniques increase the depth of penetration of the anti-bacterial agents, complete penetration, to entire length of the dentinal tubules, may not be achieved.

The present subject matter relates to systems, devices, and methods for controlling motion and causing movement of magnetically-driven microscopic particles into dentinal tubules of a tooth of a patient. The microscopic particles can include nanobots. Nanobots are microscopic particles having a size, for example, in a range of about 0.1 to 10 μm. The nanobots may be loaded with anti-bacterial agents or fabricated with materials having innate active-antibacterial properties, such as silver coating, chitosan, iron oxide nanoparticles, so that they can eliminate bacterial infection in a region in which they penetrate. A magnetic material, such as iron, may be integrated with the body of a nanobots to form the magnetically-driven nanobots. To control motion of nanobots, it may be subjected to a magnetic field. Since the magnetic material experiences an attractive force due to the magnetic field, the nanobots move under the influence of the magnetic field. In an example, to control the movement of the nanobots, one or more patterns of magnetic field, such as a rotating magnetic field, an oscillating magnetic field, a gradient magnetic field, and an elliptical magnetic field may be utilized either individually or in combinations.

In one example, a selection indicative of a pattern of movement of magnetically-driven nanobots to be achieved in the dentinal tubules can be received, for example, by a computing device. The selection may be provided, for example, by a medical professional. The selection may indicate an intended direction of movement of the nanobots. For example, the selection may relate to the degree of distribution within the dentinal tubules based on which the direction of movement of nanobots may be determined. In another example, the selection may indicate the exact pattern for movement of the nanobots, for example, ant-like movement. A signal indicative of a pattern of magnetic field to be produced can be generated based on the selection by the computing device. For example, when the selection is the ant-like movement, signal can be generated for causing production of an oscillating magnetic field. Based on the signal, an electrical signal can be generated by a signal generation unit to cause production of the pattern of magnetic field. For example, a signal generation unit can generate alternating current signals to cause generation of the oscillating magnetic field.

The electrical signal can be provided to a device which is adapted for placement on the head or around the tooth of the patient. The device may be, for example, a cap structure for placement around the tooth of the patient or a helmet, which can be placed around the head of the patient. The device can comprise cables and coils. For example, the device can comprise a first cable coupled to a first coil and the signal generation unit. The first cable can receive the electrical signal corresponding to the pattern of magnetic field to be generated by the first coil from the signal generation unit. In response to receiving the electrical signals, the first coil can generate the pattern of the magnetic field to drive the magnetically-driven nanobots from the pulp region of the tooth into the dentinal tubules.

Based on the electrical signal, different patterns of magnetic field can be produced. For example, an Alternating Current (AC) signal can cause the coil to produce an oscillating magnetic field to cause ant-like movement of the nanobots and a Direct Current (DC) signal can cause the coil to produce a gradient magnetic field to cause translational motion of the nanobots. Further, a plurality of coils may be used for production of more complex patterns of magnetic fields, such as, a rotating magnetic field and an elliptical magnetic field. For example, the device may comprise a second coil having an axis perpendicular to that of the first coil.

Accordingly, AC signals provided to the first coil and the second coil with a phase difference can produce the rotating magnetic field or the elliptical magnetic field. In an example, a third coil may be provided where an axis of the third coil is perpendicular to that of the first coil and the second coil. Thus, the first coil, the second coil, and the third coil may be provided such that the respective axes are parallel to the x-axis, y-axis, and z-axis.

With the systems, devices, and methods of the present subject matter, microscopic particles can be magnetically-driven inside microscopic channels and the motion of the microscopic particles can be controlled inside the microscopic channels, such as dentinal tubules. Thus, with the systems, devices, and methods of the present subject matter, microscopic particles including antibacterial agents can be made to penetrate a large portion of the length of the dentinal tubules. These microscopic particles may be left in the channels without any further action, or they may be driven back and recovered after the procedure. They may also be designed to degrade after some time. Thus, antibacterial agents can be delivered deep inside the dentinal tubules, enabling elimination of bacteria. Further, direction and length of penetration can be precisely controlled. Further, the magnetically-driven nanobots can be introduced into the dentinal tubules with precise spatial control over directionality and depth of penetration. The device may be implemented as a cap structure which can be placed around a tooth to be treated or a helmet which can be placed around the head of the patient. The cap structure helps in providing better control of the magnetic field and localization of treatment within the tooth. The helmet helps in treatment of multiple teeth at the same time.

The above and other features, aspects, and advantages of the subject matter will be better explained with regard to the following description and accompanying figures. It should be noted that the description and figures merely illustrate the principles of the present subject matter along with examples described herein and, should not be construed as a limitation to the present subject matter. It is thus understood that various arrangements may be devised that, although not explicitly described or shown herein, embody the principles of the present disclosure. Moreover, all statements herein reciting principles, aspects, and examples thereof, are intended to encompass equivalents thereof. Further, for the sake of simplicity, and without limitation, the same numbers are used throughout the drawings to reference like features and components.

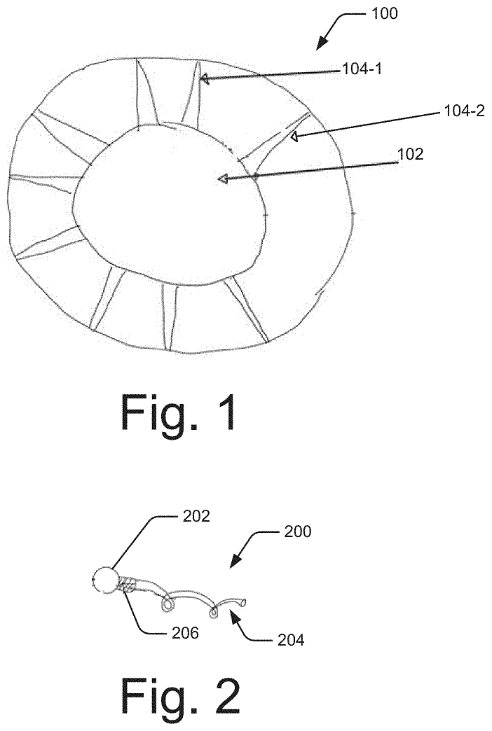

FIG. 1 illustrates a horizontal section of a sample 100 of a tooth, in accordance with an implementation of the present subject matter. The tooth may be a human tooth. The sample may have a thickness of about 200 μm and may be taken such that a central root canal space and surrounding dentine tissue is exposed. The sample 100 illustrates a pulp chamber 102, also referred to hereinafter as pulp region 102, at the centre surrounded by a plurality of dentinal tubules 104-1, 104-2, . . . Each dentinal tubule may be individually referred to as a dentinal tubule 104. The length of each dentinal tubule 104 may be in a range of about 1000 μm to 2000 μm. The length of the dentinal tubule 104 may be interchangeably referred to as depth of the dentinal tubule 104. Further, as illustrated, each dentinal tubule 104 has a tapered and cylindrical structure, with its diameter at an end near the pulp chamber 102 being larger than that at an end away from the pulp chamber 102. In an example, the diameter of the dentinal tubule 104 at the end near the pulp chamber 102 may be about 2.5 μm and the diameter of the dentinal tubule 104 at the end away from the pulp chamber 102 may be about 0.9 μm.

Due to the above special geometrical profile of the dentinal tubules, bacteria residing deep inside the dentinal tubules are inaccessible to conventional antibacterial treatment protocols. The present subject matter enables eliminating the bacteria residing deep inside the dentinal tubules with the help of nanobots that can penetrate along a large portion of the depth of the dentinal tubules.

FIG. 2 illustrates an example magnetically-driven nanobot 200, in accordance with an implementation of the present subject matter. While FIG. 2 depicts an example structure and shape of the nanobot 200, other shapes such as cylinder, ellipsoid, bent rod, curved rod, or helix are also possible. With reference to FIG. 2, the magnetically-driven nanobot 200 may include a body 202 and a tail 204. The body 202 may be spherical in shape and may be coated with anti-bacterial agents, which can eliminate the bacteria inside the dentinal tubules. The shape of the body 202 may be customized for better drug loading. Further, the tail 204 may be helical in shape, and may be alternatively referred to as a helical tail 204. The helical shape of the tail 204 facilitates in easier directed navigation of the nanobot 200.

The magnetically-driven nanobot 200 may be fabricated using a physical vapor deposition technique known as Glancing Angle Deposition (GLAD). Other shapes such as cylinder, ellipsoid, bent rod and curved rod may be fabricated using various techniques like physical vapor deposition, hydrothermal methods, chemical deposition. Each fabrication may be run on a wafer with suitable seed layer. In an example, the wafer may be a standard wafer, and have dimensions of 2 cm×2 cm. The fabrication using such a wafer may yield nanobots. In an example, the magnetically-driven nanobot 200 may have a flagellar width of approximately 250 nm and length of approximately 3 μm.

A magnetic material 206, such as iron, may be integrated into the tail 204 of the nanobot 200 during growth phase of fabrication. In an example, the magnetic material 206 may be integrated such that the magnetic material is not disposed at the centre of mass of the magnetically-driven nanobot 200. Accordingly, the disposition of the magnetic material 206 may introduce an asymmetry in the weight distribution of the magnetically driven nanobot 200. Accordingly, when the nanobot 200 is subjected to a specific pattern of magnetic field while it is near a surface, such as an internal wall of the dentinal tubule 104, the magnetically-driven nanobot 200 may undergo a net displacement. This will be explained in greater detail with reference to subsequent figures.

In operation, a plurality of magnetically-driven nanobots like the magnetically-driven nanobot 200 may be sonicated, for example, in a deionized water solution, and released into a carrier media to form a fluidic suspension of nanobots. The fluidic suspension may then be injected into the pulp region 102 of a tooth. Subsequently, the magnetically-driven nanobots 200 in the fluidic suspension may be actuated using one or more patterns of magnetic field to cause equitable three-dimensional distribution of the magnetically-driven nanobots 200 through the root canal 102 into the dentinal tubules 104. The one or more patterns of magnetic field may include an oscillating magnetic field, a rotating magnetic field, a gradient magnetic field, and an elliptical magnetic field.

To cause the production of the magnetic field, a device having coils may be used. For example, the device may be a cap structure or a helmet, as will be explained below:

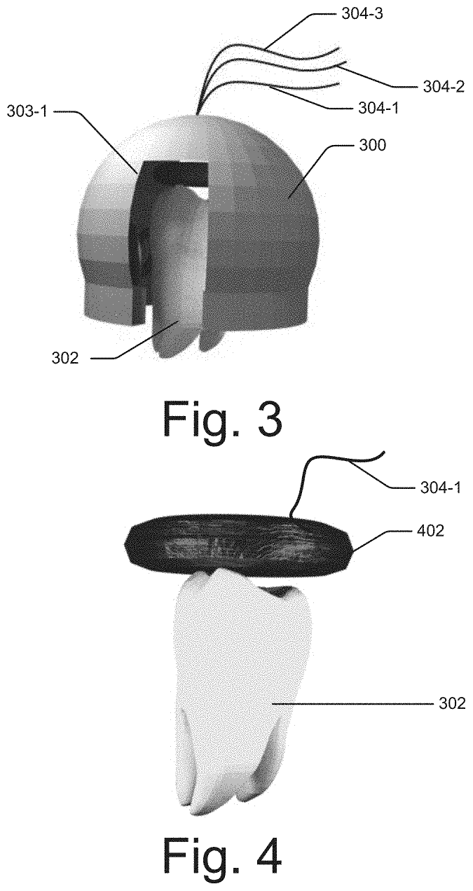

FIG. 3 depicts a cap structure 300 for causing movement of magnetically-driven nanobots into dentinal tubules of a tooth of a patient, in accordance with an example implementation of the present subject matter. The magnetically-driven nanobots may be the nanobots 200. As shown in FIG. 3(a), the cap structure 300 is adapted for placement around a tooth 302 of the patient. The dimensions of the cap structure 300 may be decided based on the tooth 302 so as to surround the tooth 302. In one example, the volume of the cap structure 300 is 1-2 cubic centimetres. The cap structure 300 can have windows 303-1, 303-2 (not shown) in opposite walls to allow placement of the cap structure 300 on the tooth 302 while making accommodation for adjacent teeth. View as illustrated in FIG. 3 depicts shows only window 303-1. It is to be understood that window 303-2 is opposite to the window 303-1.

The cap structure 300 may be fabricated from ceramic, glass, plastic, resin, or biocompatible materials. In other examples, the cap structure 300 may be fabricated from flexible biocompatible material. To cause movement of the magnetically-driven nanobots 200 into the dentinal tubules, the cap structure 300 can comprise coils (not shown) and cables 304-1, 304-2, 304-3 . . . The coils may be arranged based on the pattern of magnetic field to be produced to drive the magnetically-driven nanobots 200. The pattern of magnetic field may be a gradient magnetic field, an oscillation magnetic field, a rotating magnetic field, an elliptical magnetic field, and combinations thereof. Various coil arrangements are as explained with reference to FIGS. 4-6.

FIG. 4 depicts an example coil arrangement, in accordance with an implementation of the present subject matter. For ease in explanation, the cap structure 300 is not shown. The cap structure 300 can comprise a first coil 402. The first coil 402 may be disposed on an inner surface of the cap structure 300. The inner surface of the cap structure 300 may be the surface that faces the tooth 302. The first coil 402 may be rigid or flexible. For example, the first coil 402 may be a copper coil as shown in FIG. 4. However, for flexibility, a printed conductive membrane, such as a polyester membrane, may also be used as the first coil 402.

The first coil 402 may be disposed on the inner surface such that, in response to placement of the cap structure 300 around the tooth 302, the first coil 402 faces a side of the tooth 302. As shown in FIG. 4, the first coil 402 faces a top surface of the tooth 302. However, the first coil 402 can face any other side, for example, lateral sides of the tooth 302.

The first coil 402 can generate a pattern of magnetic field in response to receiving an electrical signal. The magnetic field generated by the first coil 402 can drive the magnetically-driven nanobots 200 (refer FIG. 2) from the pulp region 102 into the dentinal tubules 104 (refer FIG. 1). The first coil 402 can receive the electrical signal, for example, from a signal generation unit.

A first cable 304-1 can be coupled to the first coil 402 and to the signal generation unit. The first cable 304-1 can receive the electrical signal corresponding to the pattern of magnetic field to be generated by the first coil 402 from the signal generation unit. In one example, when a single coil is used in the cap structure 300, as shown in FIG. 4, the pattern of magnetic field to be generated may be an oscillating magnetic field or a gradient magnetic field. Thus, the electrical signal can be an Alternating Current (AC) signal (which causes the first coil 402 to produce the oscillating magnetic field) or a Direct Current (DC) signal (which causes first coil 402 to produce the gradient magnetic field).

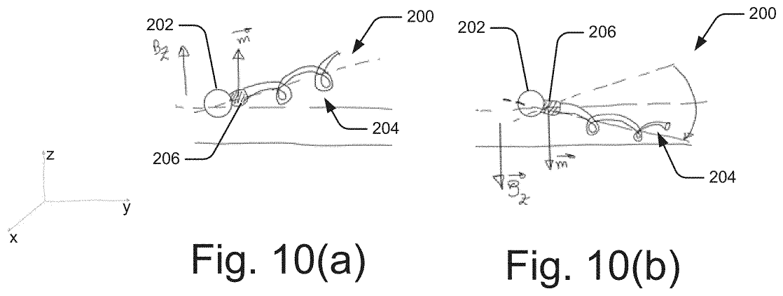

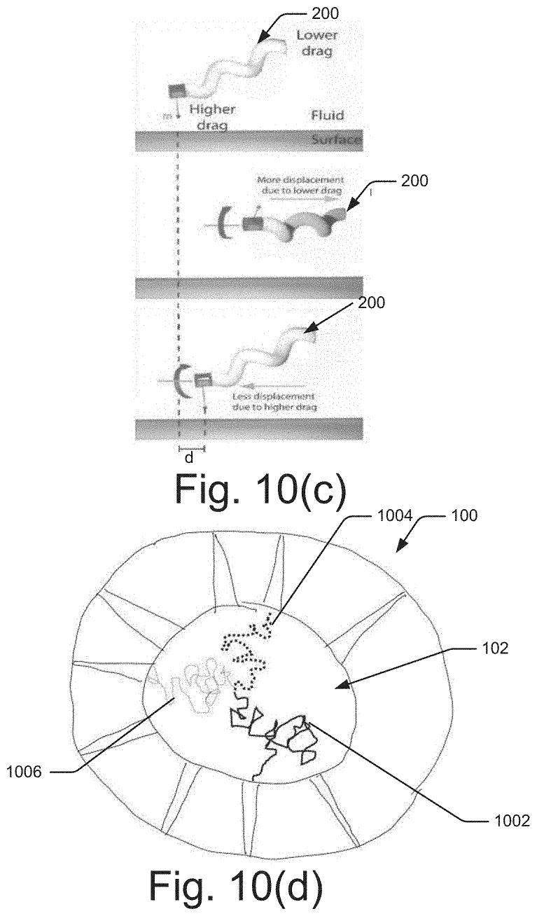

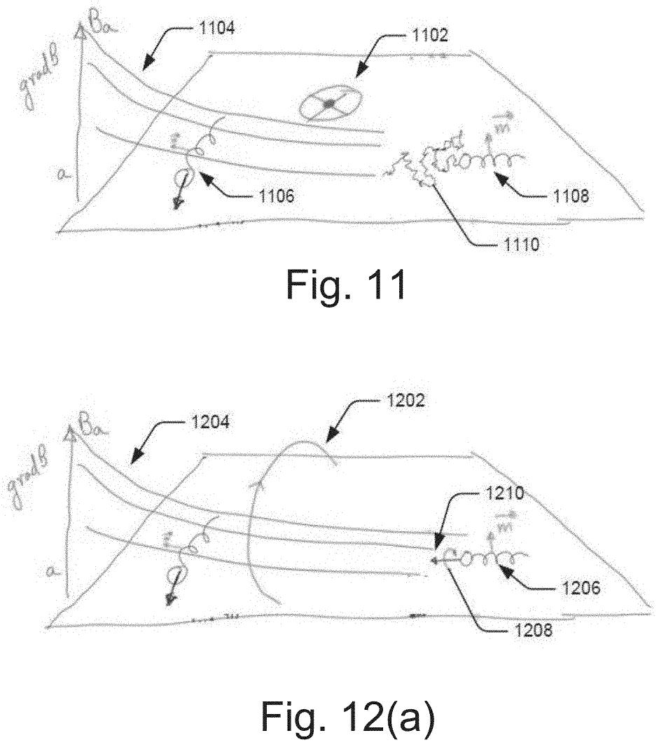

The oscillating magnetic field may refer to a magnetic field that oscillates in magnitude over time. For example, a magnitude of the magnetic field may vary in a sinusoidal pattern over time. When the oscillating magnetic field is generated the nanobots 200 can move back and forth as will be explained later with respect to FIG. 10(a)-(d). The gradient magnetic field may refer to a magnetic field which varies in strength with respect to position, for example, strength of the magnetic field may be higher at the centre of the first coil 402 and gradually decrease away from the centre. When the gradient magnetic field is generated, based on their positions, some nanobots 200 may move more strongly than other, as will be explained later with reference to FIG. 11.

In other examples, to produce more complex magnetic fields, such as a rotating magnetic field, an elliptical magnetic field, or combinations of the oscillating magnetic field, gradient magnetic field, rotating magnetic field, and elliptical magnetic field, other coil arrangements may be used.

FIG. 5 depicts another coil arrangement, in accordance with an implementation of the present subject matter. The coil arrangement, as shown in FIG. 5, comprises a first coil 502, a second coil 504, and a third coil 506. The first coil 502, the second coil 504, and the third coil 506 may be disposed on the inner surface of the cap structure 300. For the ease of explanation, the cap structure 300 is not shown in FIG. 5. Each of the first coil 502, the second coil 504, and the third coil 506 may be selected from copper coils or printed conductive membranes.

The first coil 502, the second coil 504, and the third coil 506 may be disposed such that, in response to the placement of the cap structure 300 around the tooth, the first coil 502 may face a first side of the tooth 302, the second coil 504 may face a second side of the tooth 302, and the third coil 506 may face a third side of the tooth 302. In one example, the first side corresponds to a top surface of the tooth 302, and the second side and the third side correspond to lateral surfaces of the tooth 302. The first coil 502, the second coil 504, and the third coil 506 may be disposed such that an axis of the second coil 504 is orthogonal to an axis of the first coil 502 and the third coil 506 and an axis of third coil 506 is orthogonal to axes of both the first coil 502 and the second coil 504.

Each of the first coil 502, the second coil 504, and the third coil 506 can provide a pattern of magnetic field based on an electrical signal received from the signal generation unit. The cap structure 300 can comprise the first cable 304-1, the second cable 304-2, and the third cable 304-2. The first cable 304-1, the second cable 304-2, and the third cable 304-3 can couple the first coil 502, the second coil 504, and the third coil 506, respectively, to the signal generation unit. The first cable 304-1, the second cable 304-2, and the third cable 304-3 can receive electrical signals corresponding to the pattern of magnetic field to be generated by the first coil 502, the second coil 504, and the third coil 506, respectively, from the signal generation unit.

In one example, each of the first coil 502, the second coil 504, and the third coil 506 can produce an oscillating magnetic field, for example, in response to receiving an AC signal. In another example, each of the first coil 502, the second coil 504, and the third coil 506 can produce a gradient magnetic field, for example, when a DC signal is supplied. In another example, each of the first coil 502, the second coil 504, and the third coil 506 can produce different patterns of magnetic fields. For example, the first coil 502 and the second coil 504 can produce the oscillating magnetic field while the third coil can produce the gradient magnetic field. The patterns of magnetic field to be produced by the coil may be determined based on an intended pattern of movement of the nanobots 200. The intended pattern of movement may be, for example, ant-like movement, back and forth movement, rotational movement, rocking movement, and the like.

To produce more complex magnetic fields, such as the rotating magnetic field and the elliptical magnetic field, electrical signals may be supplied to coils at a phase difference. For example, when the axis of the first coil 502 is orthogonal to the axis of the second coil 504 and AC signal is provided to the coils at a phase difference of 90 degrees, the rotating magnetic field may be generated. Further, the elliptical magnetic field can also be produced, for example, by providing AC signals of different amplitudes to the first coil 502 and the second coil 504 so that the coil with greater amplitude will produce the major axis of the elliptical magnetic field. In another examples, combinations of magnetic fields can also be produced. The combinations may be, for example, the oscillating magnetic field and the gradient magnetic field, the rotating magnetic field and the gradient magnetic field, and the like. Further, to improve strength of the magnetic field, more than three coils may also be used.

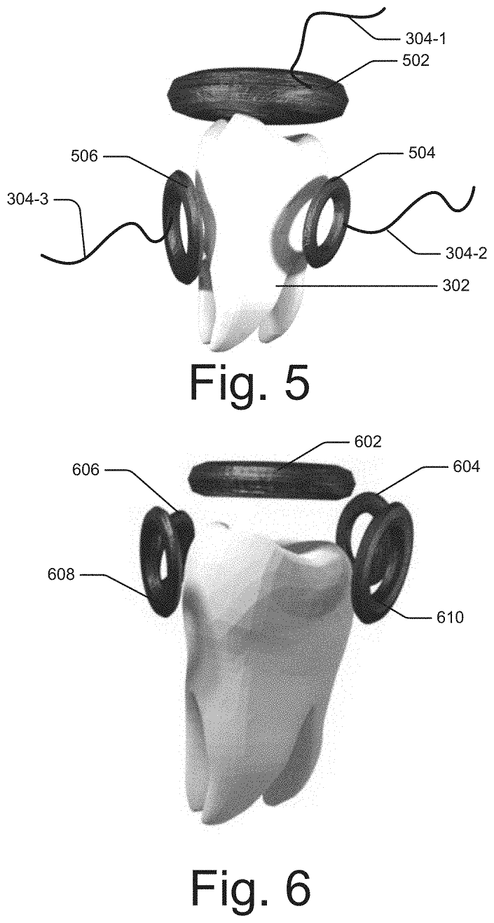

FIG. 6 depicts another coil arrangement, in accordance with an implementation of the present subject matter. The coil arrangement illustrated in FIG. 6 comprises 5 coils, namely, a first coil 602, a second coil 604, a third coil 606, a fourth coil 608, and a fifth coil 610. For ease in explanation, the cap structure 300 is not shown. The coil arrangement as shown in the FIG. 6 may be disposed on the inner surface of cap structure 300.

The coil arrangement may be disposed in the cap structure 300 such that, in response to being placed around the tooth 302, each of the coils face a side of the tooth 302. For example, as shown in FIG. 6, the first coil 602 can face a top surface of the tooth 302 and the second coil 604, the third coil 606, the fourth coil 608, and the fifth coil 610 can face a lateral surface of the tooth.

Further, the coil arrangement may be disposed such that two coils may be provided opposite each other. For example, as shown in FIG. 6, the second coil 604 may be provided opposite the fourth coil 608; and the third coil 606 may be provided opposite the fifth coil 610. In the coil arrangement as shown in FIG. 6, pairs of coils opposite to each other may be used to provide the gradient magnetic field. For example, on supply of direct current signal in opposite directions to the second coil 604 and the fourth coil 608, the gradient magnetic field may be generated. The generation of gradient fields by the opposing coils causes magnetic field of gradually decreasing strength from the coil until a centre point between the opposing coils. The other coils may be used to provide other pattern of magnetic fields, such as the oscillating magnetic field, the rotating magnetic field, the elliptical magnetic field, and combinations thereof. Such combinations of fields may help in causing different motions of the nanobots 200 depending on the field influencing the nanobots 200.

While FIG. 6 depicts that five coils may be used, any number of coils may be used. For example, 8 coils may be disposed on the inner surface of the cap structure 300. Further, while FIG. 4-6 depict that the first coil 402, 502, and 602 is provided on the top surface of the tooth, it is possible that the first coil 402, 502, 602 is provided around the lateral surface of the tooth and the top surface is free.

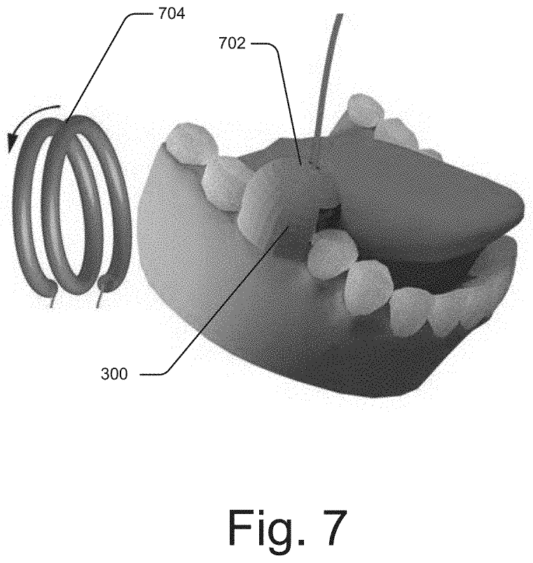

FIG. 7 depicts the cap structure 300 placed around the tooth 302, in accordance with an implementation of the present subject matter. In addition to the coil arrangement, the cap structure 300 can comprise a laser delivery unit 702. The laser delivery unit 702 may be provided on the inner surface of the cap structure 300 to deliver laser light and to cause light induced heating of the nanobots 200. In one example, the laser delivery unit 702 can comprise a laser diode disposed on the inner surface of the cap structure 300. The laser diode may be powered by an external power source to provide light to induce heat and increase the temperature of the nanobots 200 to improve antimicrobial action of the nanobots 200. In another example, the laser delivery unit 702 can comprise optical fibres which may be used instead of the laser diode to provide irradiation to the tooth 302.

In another example, the cap structure 300 may be associated with a hyperthermia coil 704. The hyperthermia coil 704 may be used in addition to or in lieu of the laser delivery unit 702 to increase the temperature of the nanobots 200. In one example, as shown in FIG. 7, the hyperthermia coil 704 may be coupled to outside the cap structure 300. FIG. 7 depicts that the hyperthermia coil 704 is provided adjacent to the tooth 302. However, the hyperthermia coil 704 may be provided on top, for example, above the head of the patient. Further, in other example, the hyperthermia coil 704 may be disposed on the inner surface of the cap structure 300. The hyperthermia coil 704 can receive high frequency alternating current to generate high frequency magnetic field. The high frequency magnetic field induces magnetic hyperthermia in the nanobots 200 to increase antibacterial efficacy of the nanobots 200 in the dentinal tubules. The hyperthermia coil 704 may be associated with a cable which can receive AC signal from the signal generation unit to generate the high frequency magnetic field.

While FIG. 7 depicts the cap structure 300 for placement around the tooth 302, other examples implementations are possible. For example, the coil arrangement to cause movement of the magnetically-driven nanobots 200 may be mounted on a helmet adapted for placement on the head of a patient.

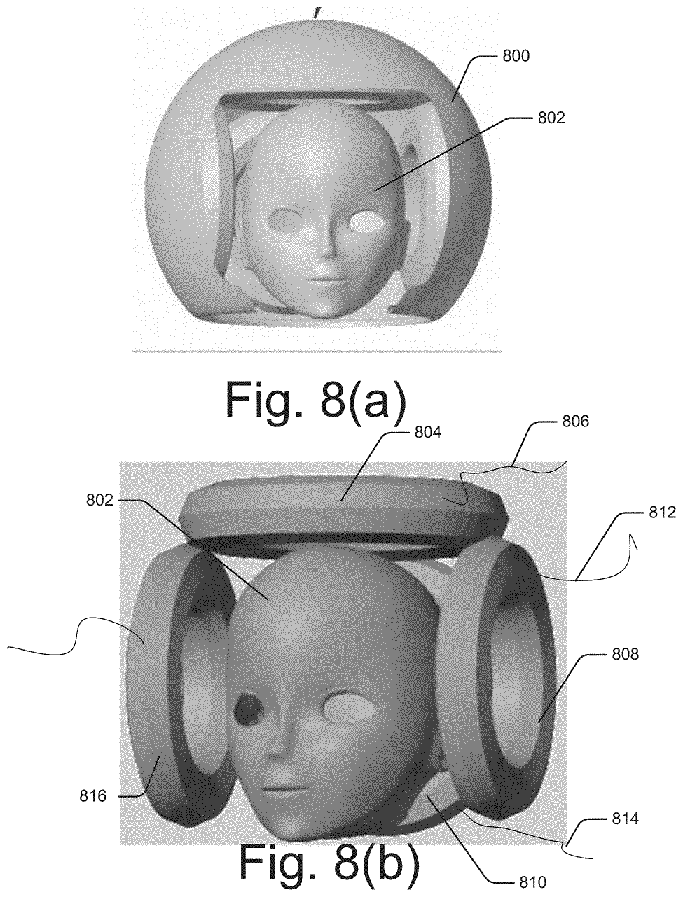

FIG. 8(a) depicts an example helmet 800 adapted for placement of the head 802 of the patient, in accordance with an implementation of the present subject matter. In one example, the volume of the helmet 800 may be decided based on the size of the head of the patient. The volume of the helmet 800 may be in a range of 2800-3200 cubic centimetres. The helmet 800 may be fabricated from non-metallic components and polymers, ceramic, glass, plastic, or the like.

FIG. 8(b) depicts a coil arrangement, in accordance with an implementation of the present subject matter. The coil arrangement may be mounted on the helmet 800. For example, the coil arrangement can be disposed on an inner surface or an outer surface of the helmet 800. For ease in explanation, the helmet 800 is not shown.

The helmet 800 can comprise a first coil 804 mounted on the helmet 800. In response to placement of the helmet 800 on the head 802 of the patient, an axis of the first coil 804 is substantially perpendicular to a first side of the tooth. The axis of the first coil 804 can be substantially perpendicular to the top surface of the tooth. The first coil 804 can generate a pattern of magnetic field in response to receiving an electrical signal, to drive the magnetically-driven nanobots 200 (refer FIG. 2) from a pulp region 102 (refer FIG. 2) of the tooth into the dentinal tubules 104.

A first cable 806 can be coupled to the first coil 804 and the signal generation unit. The first cable 806 can receive the electrical signal corresponding to the pattern of magnetic field to be generated by the first coil 804 from the signal generation unit. To produce more complex magnetic fields, such as the rotating magnetic field and the elliptical magnetic field, a plurality of coils may be provided. For example, the helmet 800 can have a second coil 808 and a third coil 810.

The second coil 808 and the third coil 810 may be mounted on the helmet 800. The second coil 808 and the third coil 810 may be arranged such that an axis of the second coil 808 is orthogonal to an axis of the first coil 804; and an axis of the third coil 810 is orthogonal to the axis of the first coil 804 and the second coil 808. A second cable 812 and a third cable 814 can couple the second coil 808 and the third coil 810, respectively, with the signal generation unit. The second cable 812 and the third cable 814 can receive the electrical signal corresponding to the pattern of magnetic field to be generated by the second coil 808 and the third coil 810, respectively.

In another example, in addition to the first coil 804, the second coil 808, and the third coil 810, other coils may be provided, for example, a fourth coil 816. The fourth coil 816 may be provided opposite to one of the other coils, for example, as shown in FIG. 8, opposite the second coil 808. The opposite pairs of coils may be used to produce the gradient magnetic field as explained with reference to FIG. 6. In addition to the first coil 804, the second coil 808, the third coil 810, the fourth coil 816, other coils may also be provided, for example, a hyperthermia coil as discussed with reference to FIG. 7. The first coil 804, the second coil 808, and the third coil 810 may be copper coils. In an example, a diameter of the first coil 804, the second coil 808, and the third coil 810 is in a range of 15-20 cm. As previously explained, the first coil 804, the second coil 808, and the third coil 810 can be used to generate the pattern of magnetic field to cause movement of magnetically-driven nanobots into dentinal tubules of the tooth.

As explained above, the electrical signal is provided to the coils to generate the pattern of magnetic field. The provision of the electrical signal may be controlled by a computing device and a signal generation unit, as will be explained below:

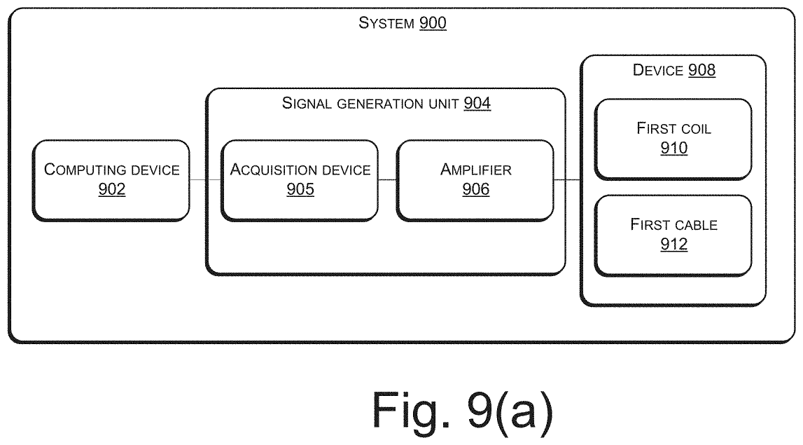

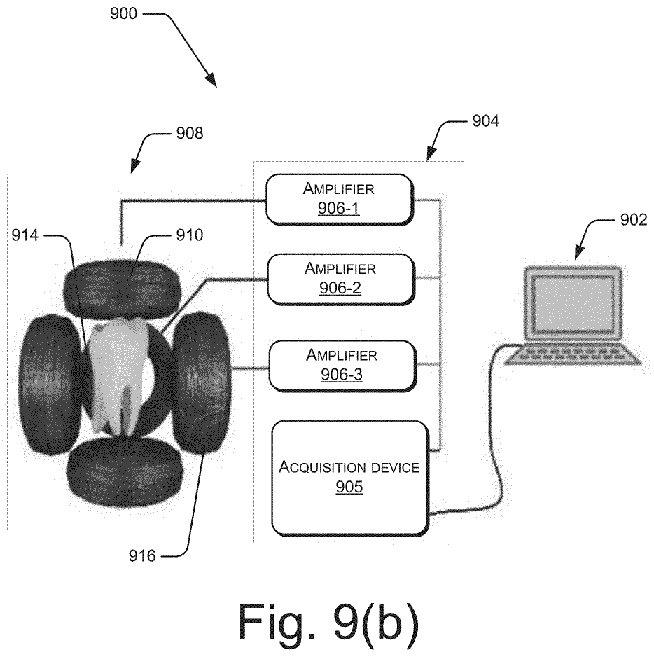

FIG. 9(a) depicts block diagram of a system 900 to cause movement of magnetically-driven nanobots into dentinal tubules of a tooth of a patient, in accordance with an example implementation of the present subject matter. The magnetically-driven nanobots may be, for example, nanobots 200. The system 900 can comprise a computing device 902, a signal generation unit 904, and a device 908. In an example, the signal generation unit 904 can comprise an acquisition device 905. In another example, the signal generation unit 904 can comprise the acquisition device 905 and an amplifier 906.

The computing device 902 may be, for example, a laptop, a personal computer (PC), a server, a tablet, a mobile device, and the like. The computing device 902 can receive a selection indicative of a pattern of movement of magnetically-driven nanobots 200 (not shown) in the dentinal tubules. The selection may be received, for example, from a user interface displayed on the computing device 902. The selection may be provided, for example, by a medical professional.

The selection may relate to intended movement of the magnetically drive nanobots 200. In another example, the selection may be angular distribution of magnetically-driven nanobots in the dentinal tubules, amplitude of magnetic field strength, area of distribution, time period of treatment, and the like. For example, the selection may be uniform angular spread or may target a sector of the tooth. In another example, the selection may be an exact pattern of movement, for example, the selection may be ant-like movement, back and forth movement, translational movement, rotational movement, and the like. In another example, the selection may be a combination of both the intended movement and the exact movement. Thus, the selection may be one of: the pattern of magnetic field to be produced, angular distribution of magnetically-driven nanobots in the dentinal tubules, amplitude of magnetic field strength, area of distribution, time period of treatment, and combinations thereof.

Based on the selection received, the computing device 902 can generate signals indicative of the pattern of magnetic field to be produced. The pattern of magnetic field may be an oscillating magnetic field, a gradient magnetic field, a rotating magnetic field, an elliptical magnetic field, and combinations thereof. For example, when uniform angular spread is selected, signals for generating oscillating magnetic field may be produced and when a sector of the tooth is to be targeted, signals for generating rotating magnetic field may be produced. For example, to produce an oscillating magnetic field, the computing device 902 can provide a plurality of digital signals that cyclically vary in values between −10 V and +10 V. For instance, a plurality of signals with gradually increasing values from −10 V to +10 V may be followed by signals gradually decreasing in value from +10 V to −10V. The computing device 902 can send the sequence of digital signal levels, in one example, may be more than 10000 levels per cycle. In another example, to produce the gradient magnetic field, the computing device 902 generates a single voltage value between −10 V and +10 V with no variation with time.

The signals may be provided to the signal generation unit 904. In one example, the signal generation unit 904 can comprise an acquisition device 905, for example, a Data Acquisition (DAQ) card which can convert the signal provided by the computing device 902 to the electrical signal. Thus, the signal generation unit 904 can generate, based on the signal received from the computing device 902, the electrical signal to cause the production of the pattern of magnetic field.

In another example, in addition to the acquisition device 905, the signal generation unit 904 can comprise an amplifier 906. In said example, the acquisition device 905 can receive the signal from the computing device 902 and generate an intermediate electrical signal to cause production of the pattern of magnetic field. The intermediate electrical signal may be an unamplified electrical signal. The amplifier 906 can receive the intermediate electrical field from the acquisition device 905, amplify the intermediate electrical field to obtain the electrical signal; and provide the electrical signal to the device 908. The amplifier 906 may thus be electrically coupled between the acquisition device 905 and the device 908.

The device 908 may be adapted for placement on the head or around a tooth of the patient. For example, the device 908 may be the cap structure 300 or the helmet 800, as described previously, which comprises coils to generate the pattern of magnetic field. In one example, as shown in FIG. 9, each coil in the device 908 may be coupled to a separate amplifier 906. Accordingly, the number of amplifiers in the system 900 may equal the number of coils in the device 908. In another example, a single amplifier 906 may be coupled to all coils of the device 908. The device 908 can comprise a first coil 910 and a first cable 912 to couple to first coil 910 to the signal generating unit 904. The first coil 910 may function similar to first coil 402, 502, 602 of the cap structure 300 or the first coil 804 of the helmet 800. The first coil 910 and the first cable 912 of the device 908 can be arranged and deployed as explained with reference to cap structure 300 in FIGS. 4-6 or the helmet 800 in FIG. 8.

FIG. 9(b) depicts an example arrangement of the system 900, in accordance with an implementation of the present subject matter. As shown in FIG. 9(b), the signal generation unit 904 comprises both the acquisition device 905 and amplifier 906. Further, there are a plurality of amplifiers 906-1, 906-2, 906-3. Each amplifier of amplifiers 906-1, 906-2, 906-3 may be coupled to a respective coil. For example, the amplifier 906-1 may be coupled to the first coil 910, the amplifier 906-2 may be coupled to a second coil 914, and the amplifier 906-3 may be coupled to a third coil 916 by respective cables. The second coil 914 may be the second coil 504, 604 of the cap structure 300 or the second coil 808 of the helmet 800. The third coil 916 may be the third coil 506, 606 of the cap structure 300 or the third coil 810 of the helmet 800. In one example, mirror opposite coils can be coupled to the sample amplifier. For example, the second coil 604 and the fourth coil 608 may be coupled to the same amplifier 906-2; and the second coil 808 and the fourth coil 816 may be coupled to the same amplifier 906-2.

In operation, with reference to FIGS. 9(a) and 9(b), the device 908 may first be calibrated. For calibration, the acquisition device 905 may first provide an electrical signal of 1 V to the amplifier 906. The amplifier 906 can amplify the electrical signal and supply it to the coils in the device 908. The magnetic field generated by the coils in the device 908 may be measured using a gaussmeter. For example, the measured magnetic field may be ‘B’. The measured magnetic field may be considered as the multiplication factor. On determining the multiplication factor, depending on the magnetic field to be produced by the coil, the electrical signal can be generated. For example, if it was found that providing 1 V to the amplifier 906 produced 1 mT magnetic field in the coils in the device 908, then to generate a magnetic field of 0.85 mT, electrical signal of 0.85 V may be supplied to the coil.

Based on the selection received by the computing device 902, the signal can be provided to the signal generation unit 904 indicative of the pattern of magnetic field to be generated. The signal generation unit 904 can then provide the electrical signals to the coils for generating the pattern of magnetic field. In one example, the pattern of magnetic field generated may be temporally varied. For example, the gradient magnetic field may be provided for a first period of time, followed by rotating magnetic field for a second period of time; or the oscillating magnetic field may be provided for a first period of time, followed by the rotating magnetic field for a second period of time, and a gradient magnetic field for a third period of time. The various magnetic field patterns are described with reference to the subsequent Fig(s).

FIGS. 10(a) and 10(b) illustrate movement of the nanobot 200 under the influence of an oscillating magnetic field, in accordance with an implementation of the present subject matter. The oscillating magnetic field can be generated by providing an AC signal to the coil. In one example, the movement of the nanobots 200 can be controlled by using only the oscillating magnetic field having frequencies of 0.1-100 Hz. In said example, the oscillating magnetic field can be generated by providing the AC signal to any one of the coils in the cap structure 300 or the helmet 800. For example, the alternating current signal may be provided to the first coil 402 of the cap structure 300 or the first coil 804 of the helmet 800.

The oscillating magnetic field may refer to a magnetic field that oscillates in magnitude over time. For example, a magnitude of the magnetic field may vary in a sinusoidal pattern over time. While the magnetic field may be directed towards a positive z-direction at a first point of time, as illustrated in FIG. 10(a), and at a second point of time, the magnetic field may be directed towards a negative z-direction, as illustrated in FIG. 10(b). Oscillating magnetic field can enhance the diffusivity of reciprocal swimmers, such as the nanobot 200, due to loss of orientation.

Since the magnetic material 206 tends to align with the magnetic field, the magnetic material 206 tends to move towards the positive z-direction at the first point of time and towards the negative z-direction at the second point of time. This is illustrated by the direction of a magnetic moment vector) in the FIGS. 10(a) and 10(b), respectively.

When a symmetrical magnetic body is subjected to an oscillating field, the body undergoes a reciprocal motion, i.e., moves back and forth between two given positions. However, when the nanobot 200 having an asymmetrical weight distribution is subjected to the oscillating magnetic field, the reciprocal motion of the tail 204 causes a net displacement of the nanobot 200, i.e., a difference in drags is introduced due to asymmetrical weight distribution in the presence of a surface nearby. Accordingly, between the first point of time and the second point of time, the nanobot 200 undergoes a translational motion. FIG. 10(c) depicts effective displacement ‘d’ of nanobot 200 under the oscillating magnetic field in the presence of a surface (like a dentinal tubule, for example), in accordance with an example implementation of the present subject matter. Such a translational motion or displacement may cause movement of the nanobot 200 into the dentinal tubule and further propagation within the dentinal tubule.

FIG. 10(d) illustrates trajectories 1002, 1004, and 1006 followed by three nanobots under the influence of the oscillating magnetic field, in accordance with an implementation of the present subject matter. The nanobots may undertake the trajectories illustrated herein in the pulp chamber 102 under the influence of the oscillating magnetic field, till they reach a respective dentinal tubule. This motion may be a reciprocal motion along with diffusion. Once a nanobot finds itself just inside a dentinal tubule, surface effects would take over in the confined space inside the dentinal tubule. Due to asymmetric interaction of the nanobot with an inner surface of the dentinal tubule and due to asymmetric weight distribution of the nanobot, the nanobot may change its movement to a back and forth rocking motion, resulting in its net displacement inside the dentinal tubule. In some examples, to amplify the rocking motion of the nanobot 200, combinations of two or more magnetic fields may be used, as will be explained below:

In an example, the oscillating magnetic field may be used in combination with a gradient magnetic field to amplify the rocking motion of the nanobot 200 inside the dentinal tubule. The gradient magnetic field may be introduced in an XY plane, for example, by providing DC signals to second coils 504, 604, 808 and third coils 506, 606, 810 of the cap structure 300 and the helmet 800 respectively. The oscillating magnetic field may be provided along a Z direction, for example, by providing AC signals to the first coils 502, 602, 804 of the cap structure 300 and the helmet 800 respectively. This causes a resultant magnetic field that forms an arc along the YZ plane.

FIG. 11 illustrates the motion of nanobots under the influence of a combination of an oscillating magnetic field 1102 and a gradient magnetic field 1104, in accordance with an implementation of the present subject matter. A gradient magnetic field may refer to a magnetic field that may increase or decrease in magnitude along space. For instance, magnetic field may be stronger near centre of the coil, and the magnetic field gradually weakens as the distance from the source increases.

The gradient magnetic field 1104 strongly binds nanobots closer to the centre of the coil producing the magnetic field, while the nanobots farther away from the centre may not be significantly impacted by the gradient magnetic field. As illustrated, the magnetic moment vector (\vec{m}) of a nanobot 1106, which is closer to the centre of the coil producing the gradient magnetic field, is aligned with the gradient magnetic field, whereas the magnetic moment vector (\vec{m}) of a nanobot 1108, which is farther from the centre of the gradient magnetic field, is not aligned with the gradient magnetic field. Accordingly, the nanobot 1106 may exhibit an ant-like motion, while the nanobot 1108 may exhibit enhanced diffusion. The direction in which the nanobot 1108 moves under the influence of the oscillating magnetic field 1102 and weak gradient magnetic field 1104 is illustrated by a pattern 1110. The pattern 1110 represents a trajectory of nanorobot exhibiting enhanced diffusion under an oscillating field.

The provision of a combination of the oscillating magnetic field 1102 and the gradient magnetic field 1104 ensures motion of nanobots in a particular region, while restricting motion of nanobots in another region. In an example, the gradient magnetic field 1104 may be provided such that nanobots in the region near the dentinal tubule 104 experiences a strong gradient magnetic field while the nanobots in the region away from the dentinal tubule 104, and near the centre of the pulp chamber 102, experiences a weak gradient field and its motion is dominated by the oscillating magnetic field. Such an arrangement ensures that the nanobots that have already entered the dentinal tubule 104 do not move out of the dentinal tubule due to an exaggerated movement of the nanobots, which may otherwise be caused due to the absence of the gradient magnetic field 1104, while the nanobots near the centre of the pulp chamber 102 can move towards the dentinal tubules through enhanced diffusion. Further, the gradient magnetic field 1104 along with the oscillating magnetic field 1102 ensures that the nanobots move inside the tubule in a rocking motion. The gradient field 1104 provides another asymmetry to the nanobots. Thus, in the presence of a surface, for example, dentinal tubules, weight asymmetry can amplify the rocking movement.

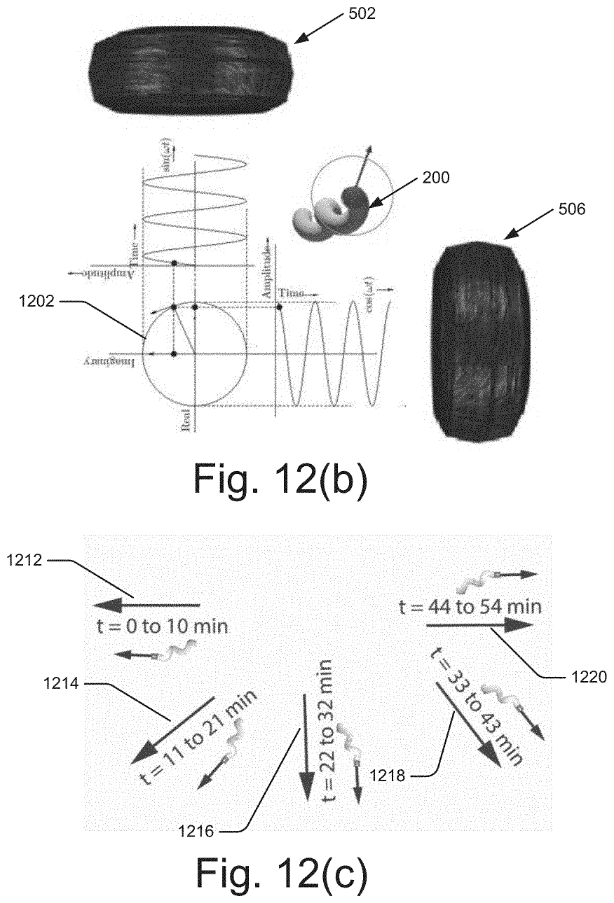

FIG. 12(a) illustrates the motion of nanobots under the influence of a combination of a rotating magnetic field 1202 and a gradient magnetic field 1204, in accordance with an implementation of the present subject matter. The gradient magnetic field 1204 may be produced, for example, by providing DC signals in opposite directions to opposite pairs of coils, such as, the second coil 604 and the fourth coil 608; or the second coil 808 and fourth coil 816 of the helmet 800.

The rotating magnetic field 1204 may be produced by coils whose axes are orthogonal to each other by providing AC signals to the coils with a phase difference of 90 degrees. For example, the rotating magnetic field can be produced by providing alternating current signals with a phase difference of 90 degrees to first coil 502 and third coil 506 or first coil 602 and third coil 606 of the cap structure 300; or the first coil 804 and the third coil 810 of the helmet 800.

The gradient magnetic field 1204 restricts the motion of the nanobots closer to the centre of the coils, as explained earlier. Further, the nanobots farther from the centre of the coils are not impacted by the gradient magnetic field 1204 and may rotate under the influence of the rotating magnetic field 1202 and move in an orthogonal direction. For instance, a nanobot 1206, which is farther from the gradient magnetic field 1204 may rotate about its long axis, as illustrated by arrow 1208, due to the influence of the rotating magnetic field 1202. This will cause it to move forward along the direction depicted by the arrow 1210. This is explained further with reference to FIG. 12(b).

FIG. 12(b) depicts motion of the nanobots under the influence of the rotating magnetic field, in accordance with an example implementation of the present subject matter. For sake of explanation, orthogonal first coil 502 and third coil 506 have been used. When AC signal is supplied to the first coil 502 and the third coil 506 at a phase difference of 90 degrees, the rotating magnetic field 1202 is generated. With reference to FIG. 12(b), when the axis of the first coil 502 is parallel to the z-axis and the axis of the third coil 506 is parallel to the x-axis, the plane of rotation of the rotating magnetic field 1202 is the XZ plane. Under the influence of the rotating magnetic field 1202, the nanobot shown as inset may move perpendicular to the plane of rotation. For example, the nanobot can move along the y-axis.

Since a rotating magnetic can only deliver the nanobots along one direction, revolving the plane of rotating field can increase overall distribution and penetration. With reference to FIG. 12(c), revolving the plane of rotation may be achieved by moving the rotating magnetic field along each angle for a pre-determined period of time. The angle may be varied by changing the phase difference of AC signal provided to orthogonal coils. For example, the rotating magnetic field may be created perpendicular to arrow 1212 from 0 to 10^{th} minute, perpendicular to arrow 1214 from 11^{th} to 21^{st} minute, perpendicular to arrow 1216 from 22^{nd} to 32^{nd} minute, perpendicular to arrow 1218 from 33^{rd} to 43^{rd} minute, and perpendicular to arrow 1220 from 44^{th} to 54th minute.

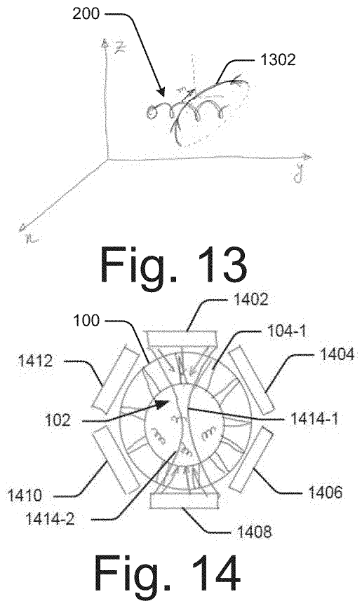

FIG. 13 illustrates motion of a nanobot 200 under the influence of an elliptical magnetic field 1302, in accordance with an implementation of the present subject matter. As illustrated, the elliptical magnetic field 1302 may be at an angle with the XZ plane. To generate elliptical magnetic field 1302 at an angle with the XZ plane, in one example, with reference to FIG. 5, the first coil 502 may be supplied with AC signal having amplitude twice as compared to the second coil 504. Thus, the magnetic field strength generated by the first coil 502 is longer when it is aligned towards first coil 502 but shorter when it rotates and aligns towards the second coil 504. Thus, a rotating magnetic field is generated but the rotating magnetic field does not rotate with equal radius all the time. Thus, the rotation is about an ellipse, forming the elliptical magnetic field 1302.

The elliptical magnetic field 1302 and the asymmetric weight distribution of the nanobot 200 can induce ant-like motion in presence of a surface, such as an internal wall of the dentinal tubule. For instance, the elliptical magnetic field 1302, which is tilted with respect to the XY plane, causes head of the nanobot 200 to wobble in an up and down manner. In such a case, one end of the nanobot 200 may have more interaction with a surface of a dentinal tubule compared to another end of the nanobot 200. This causes a net displacement of the nanobot in an ant-like motion.

FIG. 14 illustrates subjecting nanobots to a combination of an oscillating magnetic field and a gradient magnetic field, in accordance with an implementation of the present subject matter. As illustrated, the sample 100 may surrounded by a plurality of coils 1402-1412. The plurality of coils 1402-1412 can be part of the cap structure 300 or the helmet 800. As shown in FIG. 14, all the coils 1402-1412 may be provided around the lateral surface of the tooth and the top surface may be free. Of the coils 1402-1412, the coils 1402 and 1408 may be supplied with a DC current, causing a magnetic field represented by the lines 1414-1, 1414-2, . . . The magnetic field may be strong in a region near the coils 1402 and 1408 and may be weaker in other regions. Accordingly, a gradient magnetic field is provided by the coils 1402 and 1408. In addition, an oscillating magnetic field (not shown in FIG. 14) may also be introduced in the vicinity of the sample 1400.

The oscillating magnetic field may be provided by the coils 1404 and 1412 and 1406 and 1410.

It may be ensured that nanobots near the dentinal tubules follow a deterministic path, as the gradient magnetic field is weak near the dentinal tubules, while the nanobots far way (e.g., >100 μm) in the pulp chamber 102 do not respond to the rotating magnetic field.

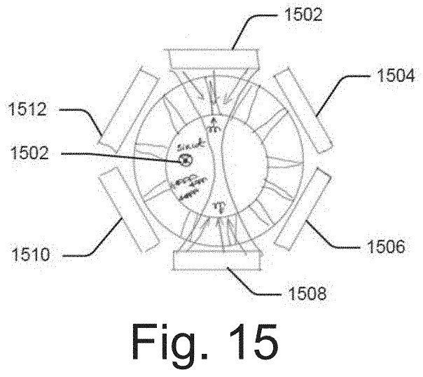

FIG. 15 illustrates the movement of nanobots under the influence of a combination of an oscillating magnetic field and a gradient magnetic field, in accordance with an implementation of the present subject matter. Here, the coils 1502 and 1508 may provide a gradient magnetic field, while the coils 1504 and 1510 provide an oscillating magnetic field. The coils 1506 and 1512 may be maintained at an off state. The combination of the gradient magnetic field provided by the coils 1502 and 1508 and oscillating magnetic field provided by the coils 1504 and 1510 causes localized rotating magnetic fields, as illustrated by the rotating magnetic field.

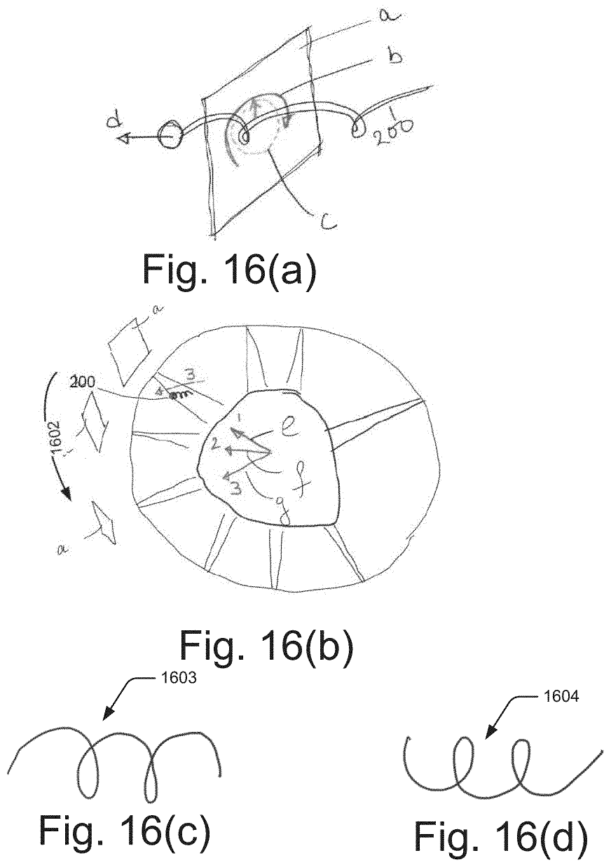

FIG. 16(a) illustrates movement of the nanobot 200 under the influence of a slowly changing rotating magnetic field, in accordance with an implementation of the present subject matter. A rotating magnetic field ‘b’ will cause the nanobot 200 to propel forward in a direction ‘d’, perpendicular to a plane ‘a’ of the rotating magnetic field V.

FIG. 16(b) illustrates variation of the rotating magnetic field ‘b’ with time, in accordance with an implementation of the present subject matter. The direction of the magnetic field ‘b’ at three different points of time are illustrated by ‘e’, ‘f’, ‘g’. Accordingly, the plane ‘a’, in which the rotating magnetic field rotates, rotates in a direction illustrated by the arrow 1602.

Such a magnetic field causes the nanobot 200 that went inside a dentinal tubule when the direction was along ‘e’ to align with direction along ‘g’ at a third point of time. On encountering the internal wall of the dentinal tubule, due to interaction with the internal wall, the nanobot 200 sticks to the internal wall and becomes immobile. This prevents the nanobot 200 from coming out of the dentinal tubule when a magnetic field is applied in the opposite direction. Thus, the present subject matter ensures that the nanobots are delivered inside the dentinal tubules and are retained inside the dentinal tubules. Such a technique may be used in conjunction with nanobots of different handedness, such as a right-handed nanobot 1603 shown in FIG. 16(c) and a left-handed nanobot 1604 shown in FIG. 16(d), so that during application of magnetic field in one direction, the right-handed nanobot 1603 follows the magnetic field and the left-handed nanobot 1604 moves in direction opposite the magnetic field.

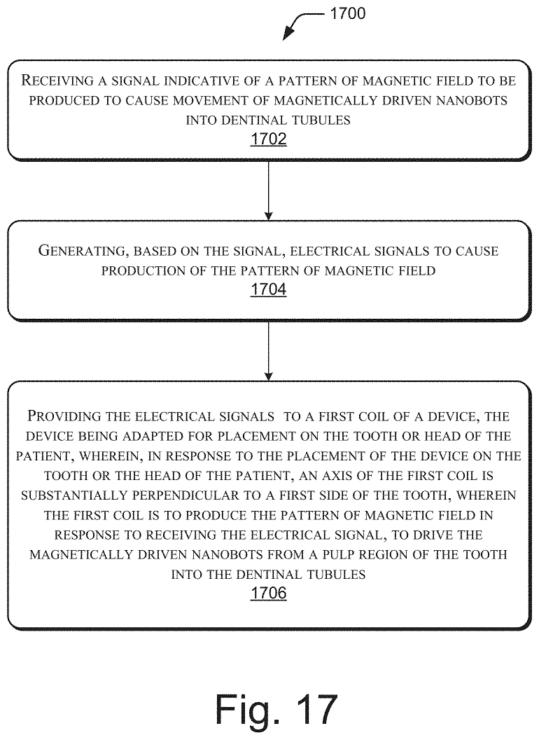

FIG. 17 depicts a method 1700 for causing movement of magnetically-driven nanobots comprising an integrated magnetic material into dentinal tubules of a tooth of a patient, in accordance with an implementation of the present subject matter. The order in which the method 1700 is described is not intended to be construed as a limitation, and any number of the described method blocks can be combined in any order to implement method 1700 or an alternative method. Additionally, individual blocks may be deleted from the method 1700 without departing from the spirit and scope of the subject matter described herein. Furthermore, the method 1700 may be implemented in any suitable hardware, computer readable instructions, firmware, or combination thereof. For discussion, the method 1700 is described with reference to the implementations illustrated in FIGS. 1-16.

A person skilled in the art will readily recognize that blocks of the method 1700 can be performed by programmed computers. Herein, some examples are also intended to cover program storage devices and non-transitory computer readable medium, for example, digital data storage media, which are computer readable and encode computer-executable instructions, where said instructions perform some or all of the blocks of the described method 1700. The program storage devices may be, for example, digital memories, magnetic storage media, such as magnetic disks and magnetic tapes, hard drives, or optically readable digital data storage media.

The method 1700, at block 1702, comprises receiving a signal indicative of a pattern of magnetic field to be produced to cause movement of magnetically-driven nanobots into dentinal tubules. The signal may be received by the signal generation unit 904 from the computing device 902. The signal may be produced based on a selection indicative of the pattern of movement of magnetically-driven nanobots 200. The selection may be one of: the pattern of magnetic field to be produced, angular distribution of magnetically-driven nanobots in the dentinal tubules, amplitude of magnetic field strength, area of distribution, time period of treatment, and combinations thereof

At block 1704, the method 1700 comprises generating, based on the signal, electrical signals to cause production of the pattern of magnetic field. The electrical signals can be generated by the signal generation unit 904. In one example, the acquisition device 905 of the signal generation unit can generate the electrical signals. In another example, the electrical signals generated by the acquisition device 905 can also be amplified to generate amplified electrical signals.

At block 1706, the method 1700 comprises providing the electrical signals to a first coil of a device. The electrical signals may be electrical signals provided by the acquisition device 905 or amplified electrical signals provided by the amplifier 906. The device may be device 908 which may be adapted for placement on the tooth or the head of the patient. The device may be the cap structure 300 or the helmet 800. The first coil may be, for example, the first coil 304 (as shown in FIG. 3) or the first coil 804 (as shown in FIG. 8). In response to the placement of the device on the tooth of the head of the patient, an axis of the first coil is substantially perpendicular to a first side of the tooth. The first coil can produce the pattern of magnetic field in receiving the electrical signal from the signal generation unit 904. The magnetic field produced by the first coil can drive the magnetically-driven nanobots 200 from the pulp region 102 of the tooth into the dentinal tubules 104. When a single coil is used, an alternating current signal can cause the coil to produce an oscillating magnetic field and a direct current signal can cause the coil to produce a gradient magnetic field.

To produce more complex magnetic fields, such as the rotating magnetic field and the elliptical magnetic field, the device may comprise additional coils, for example, a second coil and a third coil. The second coil may be, for example, the second coil 504, the second coil 604 or second coil 808 as described with reference to FIGS. 5, 6, and 8, respectively. The third coil may be, for example, the third coil 506, the third coil 606, or the third coil 810 as described with reference to FIGS. 5, 6, and 8, respectively. To produce the pattern of magnetic field, electrical signals can be provided to the first coil, the second coil, the third coil or combinations thereof.

The present subject matter will now be illustrated with working examples, which are intended to illustrate the working of disclosure and not intended to be taken restrictively to imply any limitations on the scope of the present disclosure. Unless defined otherwise, all technical and scientific terms used herein have the same meaning as commonly understood to one of ordinary skill in the art to which this disclosure belongs. It is to be understood that this disclosure is not limited to the particular methods and experimental conditions described, as such methods and conditions may vary depending on the process and inputs used as will be easily understood by a person skilled in the art.

### EXAMPLES

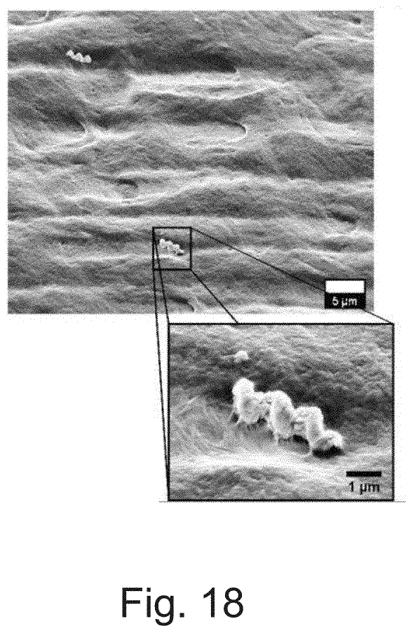

After continuous actuation, a nanobot was observed as entering a targeted dentinal tubule. It was also observed that the trajectory of the nanobot was occasionally aligning itself to the tortuous geometry of the dentinal tubule. After about 10 minutes, the nanobot was digitally gauged to have penetrated about 700 μm into the dentinal tube, away from the root canal space, from where it started. FIG. 18 depicts a Scanning Electron Microscopy (SEM) of a section of the tooth, in accordance with an example implementation of the present subject matter. From the SEM image of FIG. 18, it can be observed that the nanobots have moved into the dentinal tubules. Thus, it can be concluded that the present subject matter provides an effective method for precisely manoeuvring nanometric scale subjects inside the dentinal tubules.

Table 1 provided below example patterns of magnetic fields which were used and the results distribution of the nanobots in the dentinal tubules. Table 1 is not be construed as limiting and other patterns and combinations are possible.

TABLE 1
Example patterns of magnetic field and results

| | | | TIME PERIOD | |
|---|---|---|---|---|
| TYPE OF | | AREA OF | NEEDED FOR | |
| MAGNETIC | ANGULAR | DENTIN | AREA | |
| FIELD | DISTRIBUTION | COVERED | COVERAGE | COMMENTS |
| Unidirectional | Gaussian with | ~11% | 20 minutes | Precisely |
| rotating field | single peak | | | targeted |
| (Single | and full width | | | penetration in |
| chirality) | at half | | | one sector of |
| | maximum | | | the dentine |
| | (FWHM) ~40^{D} | | | |
| Unidirectional | Bimodal | ~28% | 20 minutes | Precisely |
| rotating field | FWHM ~50^{D} | | | targeting |
| (Opposite | | | | diametrically |
| chirality) | | | | opposite |
| | | | | sectors of the |
| | | | | dentine |
| Revolving | Uniform | ~100% | 120 minutes | Uniform |
| rotating field | distribution | | | sector by |
| | | | | sector |
| | | | | delivery in |
| | | | | dentine |
| Oscillating | Uniform | ~100% | 20 minutes | Uniform one |
| field | distribution | | | shot delivery |
| | | | | in dentine |

The present subject matter provides techniques for control of motion of microscopic particles, such as nanobots, inside dentinal tubules. Using the techniques of the present subject matter, depth of penetration of the microscopic particles inside the dentinal tubules can be accurately controlled. Further, microscopic particles can be made to reach a significant portion of the length of the dentinal tubules. Further, by using various patterns of magnetic fields and by using combinations of the patterns of the magnetic fields, accurate control of microscopic particles in different regions of a pulp chamber and dentinal tubules can be achieved.

Although the present subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. For instance, although the present subject matter is explained with reference to dentinal tubules and controlling motion of nanobots inside the dentinal tubules, the techniques of the present subject matter can be utilized for any type of dentinal tubules. Further, the motion of various types of magnetically-driven microscopic particles can be controlled using the techniques of the present subject matter.

1. A cap structure adapted for placement around a tooth of a patient to cause movement of magnetically-driven nanobots into dentinal tubules of the tooth of the patient, the cap structure comprising:
a first coil disposed on an inner surface of the cap structure, wherein, in response to placement of the cap structure around the tooth, the first coil is to face a first side of the tooth, wherein the first coil is to generate a pattern of magnetic field in response to receiving an electrical signal, to drive the magnetically-driven nanobots from a pulp region of the tooth into the dentinal tubules; and
a first cable coupled to the first coil and to a signal generation unit, wherein the first cable is to receive electrical signals corresponding to the pattern of magnetic field to be generated by the first coil from the signal generation unit.

2. The cap structure as claimed in claim 1, comprising:
a second coil disposed on the inner surface of the cap structure, wherein an axis of the second coil is orthogonal to an axis of the first coil, wherein, in response to placement of the cap structure around the tooth, the second coil is to face a second side of the tooth;
a third coil disposed on the inner surface of the cap structure, wherein an axis of the third coil is orthogonal to the axis of the first coil and the second coil, wherein, in response to placement of the cap structure around the tooth, the third coil is to face a third side of the tooth;
a second cable coupled to the second coil and the signal generation unit, wherein the second cable is to receive electrical signals corresponding to a pattern of magnetic field to be generated by the second coil from the signal generation unit; and
a third cable coupled to the third coil and the signal generation unit, wherein the third cable is to receive electrical signals corresponding to a pattern of magnetic field to be generated by the third coil from the signal generation unit.

3. The cap structure as claimed in claim 2, wherein the first coil, second coil, and the third coil are selected from:
copper coils and printed conductive membranes.

4. The cap structure as claimed in claim 1, wherein a volume of the cap structure is 1-2 cubic centimetres.

5. The cap structure as claimed in claim 1, comprising a laser delivery unit provided on an inner surface of the cap structure to deliver laser light and to cause light induced heating of the magnetically-driven nanobots.

6. The cap structure as claimed in claim 1, comprising a hyperthermia coil to receive high frequency alternating current to generate high frequency magnetic field, wherein the high frequency magnetic field induces hyperthermia in the magnetically-driven nanobots.

7. A helmet adapted for placement on the head of a patient to cause movement of magnetically-driven nanobots into dentinal tubules of a tooth of the patient, the helmet comprising:
a first coil mounted on the helmet, wherein, in response to placement of the helmet on the head of the patient, an axis of the first coil is substantially perpendicular to a first side of the tooth, wherein the first coil is to generate a pattern of magnetic field in response to receiving an electrical signal, to drive the magnetically-driven nanobots from a pulp region of the tooth into the dentinal tubules; and
a first cable coupled to the first coil and a signal generation unit, wherein the first cable is to receive electrical signals corresponding to the pattern of magnetic field to be generated by the first coil from the signal generation unit.

8. The helmet as claimed in claim 7, wherein the helmet comprises:
a second coil mounted on the helmet wherein an axis of the second coil is orthogonal to an axis of the first coil;
a third coil mounted on the helmet, wherein an axis of the third coil is orthogonal to the axis of the first coil and the second coil;
a second cable coupled to the second coil and the signal generation unit, wherein the second cable is to receive electrical signals corresponding to a pattern of magnetic field to be generated by the second coil from the signal generation unit; and
a third cable coupled to the third coil and the signal generation unit, wherein the third cable is to receive electrical signals corresponding to a pattern of magnetic field to be generated by the third coil from the signal generation unit.

9. The helmet as claimed in claim 8, wherein the first coil, the second coil, and the third coil are copper coils, wherein a diameter of the first coil, the second coil, and the third coil is in a range of 15-20 cm.

10. The helmet as claimed in claim 7, wherein a volume of the helmet is in a range of 2800-3200 cubic centimetres.

11. A system to cause movement of magnetically-driven nanobots comprising an integrated magnetic material into dentinal tubules of a tooth of a patient, the system comprising:
a computing device to:
receive a selection indicative of a pattern of movement of magnetically-driven nanobots in the dentinal tubules; and
generate a signal indicative of a pattern of magnetic field to be produced based on the selection;
a signal generation unit to:
receive the signal from the computing device; and
generate, based on the signal, electrical signals to cause production of the pattern of magnetic field; and
a device for placement on the head or around a tooth of the patient, wherein the device comprises:
a first cable coupled to a first coil and to the signal generation unit, wherein the first cable is to receive electrical signals corresponding to the pattern on magnetic field to be generated by the first coil from the signal generation unit; and
the first coil, wherein, in response to placement on the head or the tooth of the patient, an axis of the first coil is substantially perpendicular to a first side of the tooth, wherein first coil is to generate the pattern of the magnetic field in response to receiving the electrical signal, to drive the magnetically-driven nanobots from a pulp region of the tooth into the dentinal tubules.

12. The system as claimed in claim 11, wherein the single generating unit comprises:
an acquisition device to:
receive the signal from the computing device; and
generate, based on the signal, an intermediate electrical signal to cause production of the pattern of magnetic field; and
an amplifier to:
receive the intermediate electrical signal from the acquisition device;
amplify the intermediate electrical signal to obtain the electrical signal; and
provide the electrical signal to the device.

13. The system as claimed in claim 11, wherein the selection is one of: the pattern of magnetic field to be produced, angular distribution of magnetically-driven nanobots in the dentinal tubules, amplitude of magnetic field strength, area of distribution, time period of treatment, and combinations thereof.

14. The system as claimed in claim 11, wherein the pattern of magnetic field is selected from: an oscillating magnetic field, a gradient magnetic field, a rotating magnetic field, an elliptical magnetic field, and combinations thereof.

15. A method for causing movement of magnetically-driven nanobots comprising an integrated magnetic material into dentinal tubules of a tooth of a patient, the method comprising:
receiving a signal indicative of a pattern of magnetic field to be produced to cause movement of magnetically-driven nanobots into dentinal tubules;
generating, based on the signal, electrical signals to cause production of the pattern of magnetic field; and
providing the electrical signals to a first coil of a device, the device being adapted for placement on the tooth or head of the patient, wherein, in response to the placement of the device on the tooth or the head of the patient, an axis of the first coil is substantially perpendicular to a first side of the tooth, wherein the first coil is to produce the pattern of magnetic field in response to receiving the electrical signal, to drive the magnetically-driven nanobots from a pulp region of the tooth into the dentinal tubules.

16. The method as claimed in claim 15, wherein the method comprises providing electrical signals to the first coil, a second coil, a third coil or combinations thereof to produce the pattern of magnetic field in response to receiving the electrical signal, wherein on placement of the device on the head or tooth of the patient:
the second coil, wherein an axis of the second coil is orthogonal to an axis of the first coil; and
the third coil, wherein an axis of the third coil is orthogonal to the axis of the first and the third coil.

17. The method as claimed in claim 15, wherein the electrical signal is an alternating current signal or a direct current signal.
